(19) United States
(12) Patent Application Publication
(10) Pub. No.: US 2017/0035708 A1
(43) Pub. Date: Feb. 9, 2017

(54) USE OF N-(4-TERT-BUTYLBENZYL)-3-CHLORO-N-[2-(4-CHLORO-3-ETHYL-PHENYL)-ETHYL]-2-FLUORO-5-TRIFLUOROMETHYL-BENZAMIDE FOR THE TREATMENT OF EYE DISEASES

(71) Applicant: Hoffmann-La Roche Inc., Little Falls, NJ (US)

(72) Inventors: Evelyne Chesne, Sierentz (FR); Guido Hartmann, Loerrach (DE)

(73) Assignee: Hoffmann-La Roche Inc., Little Falls, NJ (US)

(21) Appl. No.: 15/091,845

(22) Filed: Apr. 6, 2016

Related U.S. Application Data
(63) Continuation of application No. PCT/EP2014/071280, filed on Oct. 6, 2014.

(30) Foreign Application Priority Data
Oct. 8, 2013 (EP) 13187678.1

(51) Int. Cl. A61K 31/166; A61K 45/06; A61K 31/047

(52) U.S. Cl. CPC A61K 31/166; A61K 31/047; A61K 45/06

(57) ABSTRACT

The present invention relates N-(4-tert-Butyl-benzyl)-3-chloro-N-[2-(4-chloro-3-ethyl-phenyl)-ethyl]-2-fluoro-5-trifluoromethyl-benz-amide which is useful in the prevention, treatment, delaying progression and/or reduction of eye diseases, in particular wherein the eye diseases are intraocular neovascular diseases and its method of preventing, retarding and ameliorating eye diseases, in particular intraocular neovascular diseases.

The present invention relates N-(4-tert-Butyl-benzyl)-3-chloro-N-[2-(4-chloro-3-ethyl-phenyl)-ethyl]-2-fluoro-5-trifluoromethyl-benz-amide which is useful in the prevention, treatment, delaying progression and/or reduction of eye diseases, in particular wherein the eye diseases are intraocular neovascular diseases and its method of preventing, retarding and ameliorating eye diseases, in particular intraocular neovascular diseases.

In another embodiment, the invention relates to N-(4-tert-Butyl-benzyl)-3-chloro-N-[2-(4-chloro-3-ethyl-phenyl)-ethyl]-2-fluoro-5-trifluoromethyl-benz-amide which is useful in the prevention, treatment, delaying progression and/or reduction of eye diseases, in particular wherein the eye diseases are proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, particularly wherein the eyes diseases are cataract, corneal clouding (opacification), age-related macular degeneration (AMD), in particular AMD, more particularly dry AMD.

In another embodiment, the invention relates to N-(4-tert-Butyl-benzyl)-3-chloro-N-[2-(4-chloro-3-ethyl-phenyl)-ethyl]-2-fluoro-5-trifluoromethyl-benz-amide's method of preventing, retarding and ameliorating eye diseases, proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, particularly wherein the eyes diseases are cataract, corneal clouding (opacification), age-related macular degeneration (AMD), in particular AMD, more particularly dry AMD.

In another embodiment, the invention comprises N-(4-tert-Butyl-benzyl)-3-chloro-N-[2-(4-chloro-3-ethyl-phenyl)-ethyl]-2-fluoro-5-trifluoromethyl-benz-amide for use as therapeutic active substances for the treatment, the prophylaxis, delaying progression and/or reduction of eye diseases, in particular wherein the eye diseases are intraocular neovascular diseases. In a particular embodiment, the present invention comprises a pharmaceutical composition comprising the N-(4-tert-Butyl-benzyl)-3-chloro-N-[2-(4-chloro-3-ethyl-phenyl)-ethyl]-2-fluoro-5-trifluoromethyl-benz-amide and a medium chain triglycerides useful for the prevention, treatment, delaying progression, and/or reduction of eye diseases, in particular wherein the eye diseases are intraocular neovascular diseases. In another embodiment of the present invention relates to composition comprising a N-(4-tert-Butyl-benzyl)-3-chloro-N-[2-(4-chloro-3-ethyl-phenyl)-ethyl]-2-fluoro-5-trifluoromethyl-benz-amide, in association with one or more carotenoid(s), in particular wherein the carotenoids are selected from xanthophylls, more particular wherein one carotenoid is lutein, most particularly wherein the carotenoids are lutein and one stereoisomer of zeaxanthin (more particularly zeaxanthin), useful for the prevention, treatment, delaying progression, and/or reduction of eye diseases, in particular wherein the eye diseases are intraocular neovascular diseases. Said composition is a nutraceutical composition or a pharmaceutical composition useful in particular for the prevention, treatment, delaying progression, and/or reduction of eye diseases in particular wherein the eye diseases are intraocular neovascular diseases.

In a particular embodiment according to the invention the eyes diseases are cataract, corneal clouding (opacification), age-related macular degeneration (AMD), in particular AMD.

### BRIEF DESCRIPTION OF THE FIGURES

FIG. 1: Viability of HUVES cells in response of VEGF under normoxia and hypoxia conditions

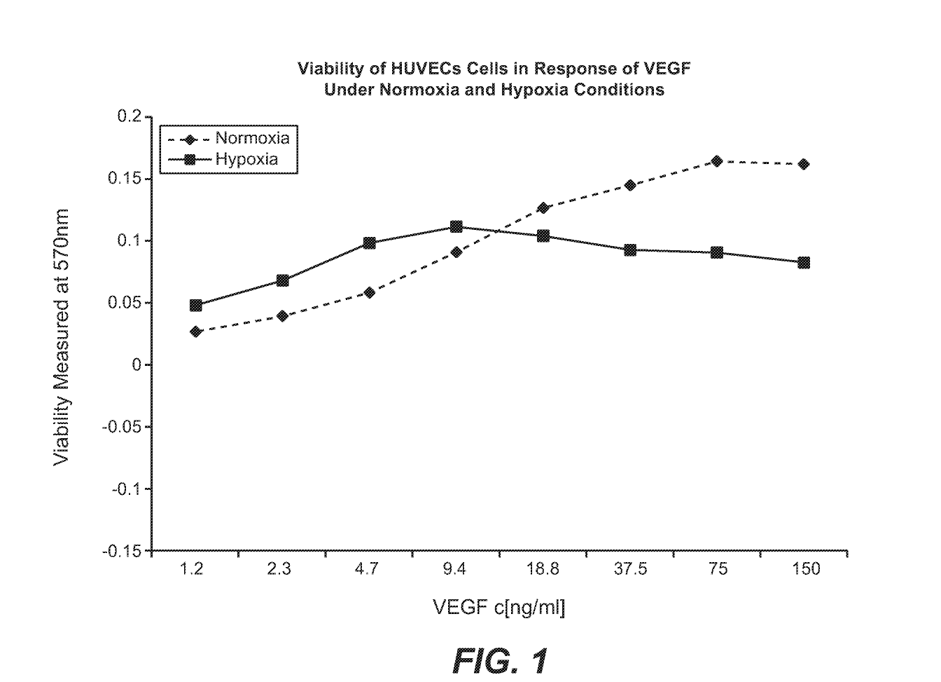

FIG. 2: Viability of HUVECS treated with CETP inhibitor under normoxia

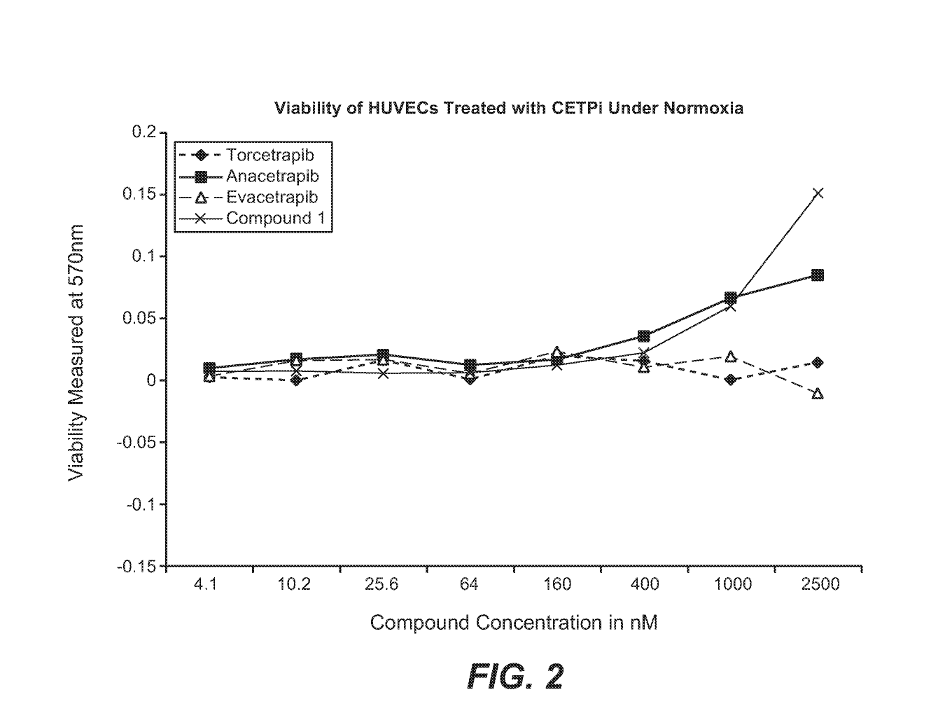

FIG. 3: Viability of HUVECs treated with CETP inhibitors under hypoxia

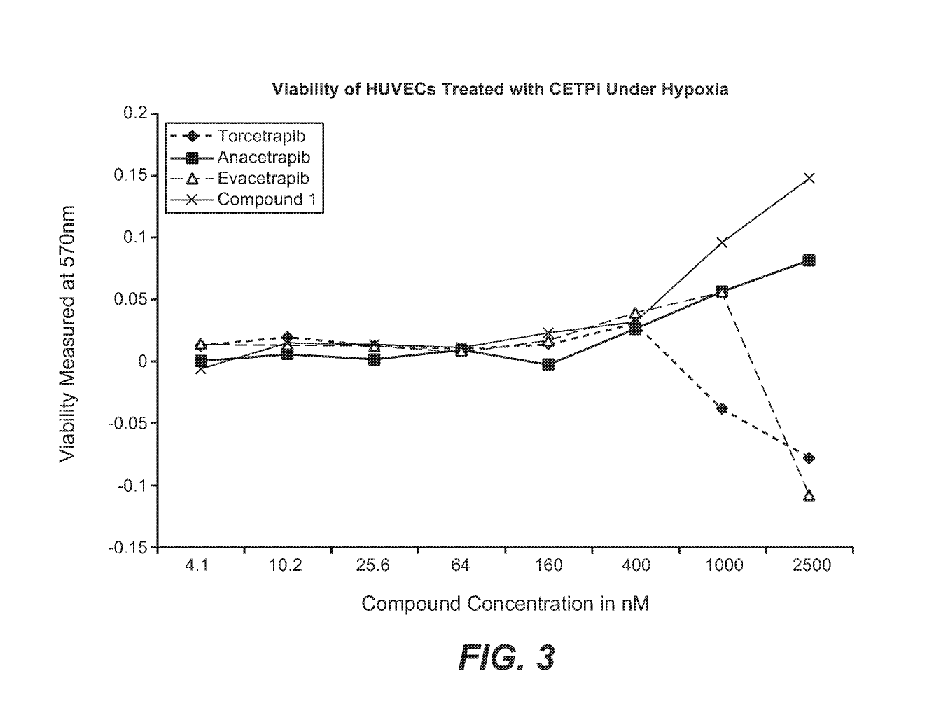

FIG. 4: illustrates a X-ray powder diffraction pattern of N-(4-tert-Butyl-benzyl)-3-chloro-N-[2-(4-chloro-3-ethyl-phenyl)-ethyl]-2-fluoro-5-trifluoromethyl-benz-amide crystalline form, also known as Form A.

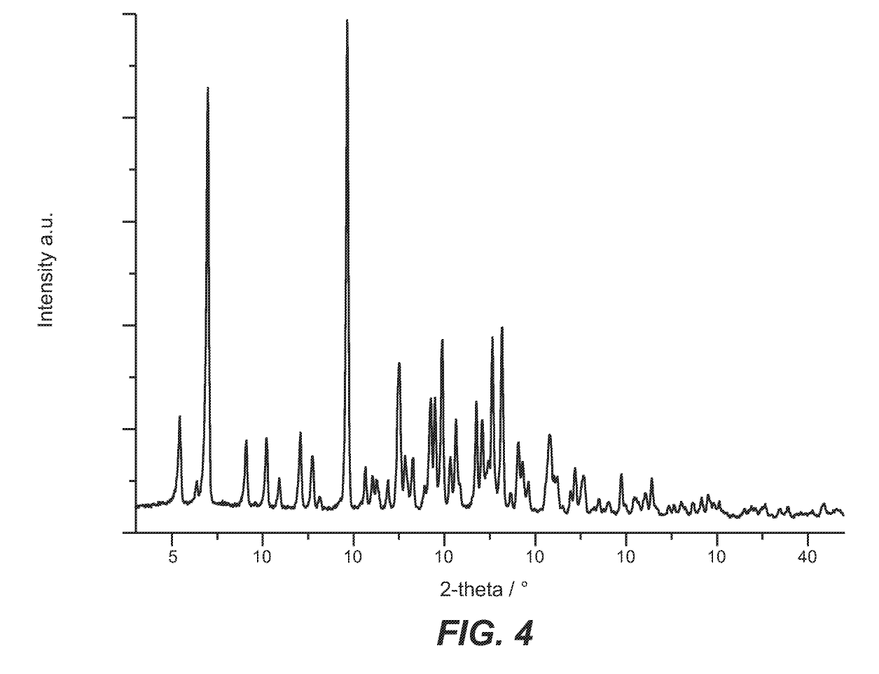

The carotenoids are naturally-occurring compounds that have antioxidant properties. The carotenoids are common compounds synthetized by plants, and contribute greatly to the colouring of plants and some animals. The large majority of animals' species of including mammals are unable to synthesize carotenoids de novo and accordingly rely upon diet to provide carotenoid requirements. Mammals also have a limited ability to modify carotenoids. A mammal can convert beta-carotene to vitamin A, but most other carotenoids are deposited in mammalian tissue in unchanged form.

With respect to humans, about ten carotenoids are found in human serum. The major carotenoids in human serum are beta-carotene, alpha-carotene, cryptoxanthin, lycopene and lutein. Small amounts of zeaxanthin, phytofluene, and phytoene are found in human organs. However, of the ten carotenoids found in human serum, only two, trans- and/or meso-zeaxanthin and lutein, have been found in the human retina. Zeaxanthin is the predominant carotenoid in the central macula or foveal region and is concentrated in the cone cells in the centre of the retina, i.e., the fovea. Lutein is predominantly located in the peripheral retina in the rod cells. Therefore, the eye preferentially assimilates zeaxanthin over lutein in the central macula which is a more effective singlet oxygen scavenger than lutein. It has been theorized that zeaxanthin and lutein are concentrated in the retina because of their ability to quench singlet oxygen and scavenge free radicals, and thereby limit or prevent photic damage to the retina.

It is known that the carotenoids other than zeaxanthin and lutein that do enter the retina may cause adverse effects, such as the formation of crystalline deposits by canthaxanthin, which may take several years to dissolve. Canthaxanthin in the retina also caused a decreased adaptation to the dark.

Vitamins (A, E or C), zinc, selenium, anthocyanins or macular pigments, such as, for example, lutein and zeaxanthin are usually used for their antioxidant properties. Lutein and zeaxanthin are macular pigments representing 99% of total pigments of the macula, which belong to the family of carotenoids, and specifically to the family of xanthophylls.

Both pigments may exhibit on one hand an indirect protective antioxidant effect through their capacity to absorb blue light particularly aggressive for photoreceptor and on the other hand direct radical scavenging antioxidant properties. US2005/0032914, US2010/0159029 and WO2009/129859 describe compositions for oral administration, comprising lutein and zeaxanthin in combination with other non-enzymatic antioxidant products, for enhancing visual performance, inhibiting macular degeneration or promoting eye health. US2007/265351 describes a xanthophyll composition comprising lutein and zeaxanthin useful for eye health.

Moreover, lutein is defined by the Afssa (Agence Francaise de Securite Sanitaire des Aliments) as “an agent which contributes to protect retina and lens against oxidation” (Saisine no 2003-SA-0205, 2004) and scientific studies have shown a relationship between the dietary intake of lutein/zeaxanthin and the likelihood of having AMD (SanGiovanni et al., AREDS report no 22, Arch Ophthalmol, 2007).

Recently, it has been demonstrated that aging eyes have a decreased amount of carotenoids deposited on the foveal region of the retina. Clinical and laboratory studies indicate that photic injury is at least one cause of age-related macular degeneration because of the cumulative effect of repeated photic insult which leads to a gradual loss of photoreceptor cells and degeneration of macular tissue.

Age-related macular degeneration (AMD) is an irreversible blinding disease of the retina. Unlike cataracts which can be restored by replacing of the diseased lens, age-related macular degeneration cannot be treated by replacing the diseased retina because the retina is a component of the central nervous system. Therefore, because no treatment for this disease exists once the photoreceptors are destroyed, prevention is the most efficient way to address age-related macular degeneration. Presently, prevention of age-related macular degeneration resides in limiting or preventing light and oxygen-induced (i.e., free radical-induced) damage to the retina because the retina is the only organ that is continuously exposed to high levels of light in a highly-oxygenated environment.

AMD is a leading cause of severe, irreversible vision loss among the elderly. Bressler, IAMA 291:1900-1 (2004). It is characterized by a broad spectrum of clinical and pathologic findings, such as pale yellow spots known as drusen, disruption of the retinal pigment epithelium (RPE), choroidal neovascularization (CNV), and disciform macular degeneration. The manifestations of the disease are classified into two forms: non exudative (dry) and exudative (wet or neovascular). Drusen are the characteristic lesions of the dry form, and neovascularization characterizes the wet form. Disciform AMD is the fibrotic stage of the neovascular lesion.

Age-related macular degeneration (AMD) is a multi-factorial, slowly-progressive, degenerative disorder of the photoreceptors in the macula that eventually leads to loss of central vision. It is generally asymptomatic during early stages of the disease (early and intermediate AMD), but late AMD (wet neovascular AMD/choroidal neovascularization [CNV] and geographic atrophy [GA]) is the leading cause of irreversible blindness among people older than 65 years in industrialized countries (The Eye Diseases Prevalence Research Group 2004). AMD accounts for 8.7% (3 million persons) of all blindness, ranging from 0% in sub-Saharan Africa to 50% in industrialized countries. More than 8 million people have at least large drusen in one eye (intermediate AMD) and 3.6 million of these have bilateral large drusen (The Eye Diseases Prevalence Research Group 2004). In Europe, the estimated prevalence of CNV is 3.3% and of GA 1.2% in those aged 65 and older (Augood C A, et al., Arch Ophthalmol. 2006; 124(4):529-535.). These prevalence figures are expected to increase by approximately 50% in the next 30 years, as a result of an ageing population. In the United States, the overall prevalence of any AMD is estimated to be 6.5% (Klein R, et al. Arch Ophthalmol. 2011; 129(1):75-80.).

Three key risk factors for developing AMD have been identified: advanced age, environmental factors and genetic susceptibility variants. Population-based studies in North America, the Netherlands and Australia identified age as the most strongly associated risk factor for AMD (Smith et al, Ophthalmol. 2001; 108(4):697-704). Smoking is a consistently associated environmental risk factor. Numerous genetic markers have been identified that are associated with the development of AMD. Several pathogenic mechanisms have been associated with AMD, such as angiogenesis (Folkman Curr Mol Med. 2003; 3(7):643-651), complement activation (Bora et al Seminar in Immunopathol. 2008; 30:85-95.; Patel M, Chan C C. Seminar in Immunopathol. 2008; 30(2):97-110.) oxidative stress (Beatty et al. Sury Ophthalmol. 2000; 45(2):115-134), and the accumulation of lipofuscin in the retinal pigmented epithelium (RPE) (Wassell et al. J Biol Chem. 1999; 274(34):23828-23832). It is believed that AMD is initiated by the malfunction of RPE cells that cannot cope with the high metabolic rate of the neuronal retina. Currently, there is no approved therapy for the treatment of early and intermediate AMD.

There is a dramatic increase in the prevalence of AMD with advancing age. See, e.g. Leibowitz et al., Sury Ophthalmol 24(Suppl):335-610 (1980) and Klein et al., Ophthalmology 99:933-43 (1992). Although the wet form of AMD is much less common, it is responsible for 80%-90% of the severe visual loss associated with AMD (Ferris et al., Arch Ophthamol 102:1640-2 (1984)). There is an estimated 1-1.2 million prevalent cases of wet AMD. The cause of AMD is unknown; however, it is clear that the risk of developing AMD increases with advancing age. Other known risk factors include family history and cigarette smoking. Postulated risk factors also include oxidative stress, diabetes, alcohol intake, and sunlight exposure. D'Amico, N Engl 1 Med 331:95-106 (1994) and Christen et al., IAMA 276:1147-51 (1996).

Dry AMD is characterized by changes in the RPE and Bruch's membrane. It is thought that the RPE, compromised by age and other risk factors, deposits lipofuscin and cellular debris on Bruch's membrane. These changes may be seen ophthalmoscopically as drusen, which are scattered throughout the macula and posterior retinal pole. There are also variable degrees of atrophy and pigmentation of the RPE. Dry AMD may be asymptomatic or accompanied by variable and usually minimal visual loss and is considered to be a prelude to development of wet AMD.

Wet AMD is typically characterized by CNV of the macular region. The choroidal capillaries proliferate and penetrate Bruch's membrane to reach the RPE and may extend into the subretinal space. The increased permeability of the newly formed capillaries leads to accumulation of serous fluid or blood under the RPE and/or the neurosensory retina or within the neurosensory retina. When the fovea becomes swollen or detached, decreases in vision occur. Fibrous metaplasia and organization may ensue, resulting in an elevated subretinal mass called a disciform scar that constitutes end-stage AMD and is associated with permanent vision loss (D'Amico D J. N Engl J Med 331:95-106 (1994)). The neovascularization in AMD can be classified into different patterns based on fluorescein angiography of subfoveal chorodial neovascular lesions. TAP and VIP Study Groups, Arch Ophthalmol 121:1253-68 (2003). The major angiographic patterns are termed classic and occult and are associated with different degrees of aggressiveness, vision losses, and response to different treatment options.

Patients with early AMD have impaired dark adaptation upon moving from a brightly lit to a dim lit room. The abnormalities in dark adaptation may precede clinically detectable macular changes earlier than any significant central field visual acuity (VA) function loss, and worsen with disease progression—attributable to further disruption of RPE. Thus, mean change from baseline in dark adaptation function was selected as the primary measure of efficacy in patients with intermediate AMD.

Population-based studies have indicated that drusen size is associated with increased risk for developing advanced AMD. Potentially through modification of plasma HDL-C and LDL-C levels by N-(4-tert-Butyl-benzyl)-3-chloro-N-[2-(4-chloro-3-ethyl-phenyl)-ethyl]-2-fluoro-5-trifluoromethyl-benz-amide, one primary direct mode of action is hypothesized to be on drusen load. Increasing drusen load between Bruch's membrane and the RPE leads to a physical separation of the RPE and choroidal vessels. This is presumed to have deleterious effects on the RPE and the overlaying neuronal retinal cells. Therefore, change in drusen volume (DV) is selected as secondary endpoint.

Currently, several AMD classification schemes, grading systems and severity scale are in use globally. There is no universally accepted definition, including initial diagnosis and staging, of the AMD phenotype for either clinical or research purposes. Recently, the Beckman Initiative for Macular Research Classification Committee (Ferris et al., J. Ophtalmol. 2013; 120(4):844-851.) re-analyzed the AREDS data in an attempt to develop a clinical classification system for AMD that is clinically usable and enables identification of patients at increased risk of developing advanced AMD. The proposed new clinical classification scale (see Table 1) is usable in most clinical settings, is consistent with skill sets of most eye care providers, and seems to be of value in predicting the risk of late AMD. This classification will be used to classify patients in this clinical study.

TABLE 1
Clinical classification of Aged Related Macular Degeneration
according to the present invention:

| | Definition |
|---|---|
| | (Lesions Assessed Within 2 Disc Diameters of |
| Classification of AMD | Fovea in either Eye) |
| No apparent aging | No drusen and No AMD pigmentary |
| changes | abnormalities^{a} |
| Normal aging changes | Only drupelets (small drusen ≦63 μm) and no |
| | AMD pigmentary abnormalities^{a} |
| Early AMD | Medium drusen >63 μm and ≦125 μm and no |
| | AMD pigmentary abnormalities^{a} |
| Intermediate AMD | Large drusen >125 μm and/or any AMD |
| | pigmentary abnormalities^{a} |
| Late AMD | Neovascular AMD and/or any GA |

AMD = age-related macular degeneration;
GA = geographic atrophy.
^{a}AMD pigmentary abnormalities: any definite hyper- or hypopigmentary abnormalities associated with medium or large drusen but not associated with known disease entities.
Data source: Ferris et al., J. Ophtalmol. 2013; 120(4): 844-851.

The patients with intermediate AMD, are at high risk of progressing to late stages of AMD. On the other hand these patients have usually good visual acuity (VA) but marked impairments in their dark adaptation function.

The group of xanthophylls includes (among many other compounds) lutein, zeaxanthin, neoxanthin, violaxanthin, and α- and β-cryptoxanthin. The latter compound is the only known xanthophyll to contain a beta-ionone ring, and thus β-cryptoxanthin is the only xanthophyll that is known to possess pro-vitamin A activity for mammals. Even then, it is a vitamin only for plant-eating mammals that possess the enzyme to make retinal from carotenoids that contain beta-ionone (some carnivores lack this enzyme). In species other than mammals, certain xanthophylls may be converted to hydroxylated retinal-analogues that function directly in vision. For example, with the exception of certain flies, most insects use the xanthophyll derived R-isomer of 3-hydroxyretinal for visual activities, which means that β-cryptoxanthin and other xanthophylls (such as lutein and zeaxanthin) may function as forms of visual “vitamin A” for them, while carotenes (such as beta carotene) do not.

Intestinal absorption and tissue distribution of carotenoids. Most likely because of their lipophilicity the absorption and distribution of carotenoids is complex and highly variable (Castenmiller, J. J. Annu Rev Nutr 1998; 18:19-38). It involves intestinal components bile acids, enzymes such as esterase, receptors in enterocytes and inclusion into lipoproteins, secretion in the plasma or lymph and distribution among lipoproteins and delivery to tissues. Greene et al (Greene, C. M. Nutr Metab (Lond) 2006; 3:-6.) noticed a relationship between lutein and zeaxanthin plasma concentration and HDL size and speculated that because of their location at the surface of lipoproteins these carotenoids could exchange during particle remodeling while HDL interact with other lipoproteins. In addition, genetic variation is involved in the inter-individual variability in carotenoid bioavailability as reviewed by Borel (Borel, P. Mol Nutr Food Res 2012 February; 56(2):228-40) who suggests a personalized dietary guideline for carotenoids according to individual genetic characteristics. Several attempts to increase the bioavailability of lutein with various formulations have been published although the availability from egg seems satisfactory the simultaneous increase in plasma cholesterol may not be appropriates in a number of patients (Thurnham, D. I. Nutr Res Rev 2007 December; 20(2):163-79).

It has been found according to the present invention that not all CETP inhibitors are useful in the prevention, treatment and/or reduction of eye diseases in particular AMD. N-(4-tert-Butyl-benzyl)-3-chloro-N-[2-(4-chloro-3-ethyl-phenyl)-ethyl]-2-fluoro-5-trifluoromethyl-benz-amide is foreseen to be useful in the prevention, treatment and/or reduction of eye diseases, in particular AMD.

It is of interest to distinguish N-(4-tert-Butyl-benzyl)-3-chloro-N-[2-(4-chloro-3-ethyl-phenyl)-ethyl]-2-fluoro-5-trifluoromethyl-benz-amide from other CETP inhibitors. Therefore there is a need to differentiate in vitro or ex vivo the partial CETP inhibitor such as N-(4-tert-Butyl-benzyl)-3-chloro-N-[2-(4-chloro-3-ethyl-phenyl)-ethyl]-2-fluoro-5-trifluoromethyl-benz-amide from all other CETP inhibitors, especially the CETP inhibitors such as 3,5-bis-trifluoromethyl-benzene derivatives (i.e. torcetrapib and anacetrapib). N-(4-tert-Butyl-benzyl)-3-chloro-N-[2-(4-chloro-3-ethyl-phenyl)-ethyl]-2-fluoro-5-trifluoromethyl-benz-amide has the potential to have beneficial clinical effects on slowing or preventing progression of intermediate AMD. The mechanism of action could be partly explained through its lipid-modulating effects on LDL-cholesterol and HDL-cholesterol, anti-oxidative and protective effects from phototoxic damage via increased delivery of lutein to the retina. Human clinical evidence of N-(4-tert-Butyl-benzyl)-3-chloro-N-[2-(4-chloro-3-ethyl-phenyl)-ethyl]-2-fluoro-5-trifluoromethyl-benz-amide-mediated beneficial effects could be demonstrated by a decrease in drusen formation, an increase in macular pigment with a positive change in the dark adaptation function of the retina.

In parallel, it has been suggested that the roles of hypoxia may be involved in the development of AMD. (O. Arjamaa et al., Ageing research Reviews 8 (2009), 349-358). Arden G., in a review article, highlights again the role of hypoxia and its role in the increase in VEGF. Studies have demonstrated the wide spread obliteration of retinal capillaries, leaving the inner retina with little blood supply (Arden G. G, et al, Doc Ophthlmol (2012)124; 15-26). The article continues on to explain the importance of the oxygen in the retina. Confirmation in animal model studies wherein VEGF antagonists provided benefits in patient with neovascular AMD (Campochiaro P., J Mol Med. (2013) 91; 311-321).

Unless otherwise stated, the following terms used in the specification and claims have the meanings given below:

As used herein, the singulars forms “a”, “an”, and “the” include plural referents unless the contents clearly dictates otherwise. Therefore a compound optionally includes a combination of two or more such compound, and the like.

“Medium chain triglycerides (MCT)” are lipophilic solubilizers. They are triglyceride mixtures of medium chain fatty acids (C6-C12). Commercial products can be further fractionated to contain mainly C6-C10 triglycerides or C8-C12 triglycerides. Usually their HLBs are less than 2. They are also commonly named “neutral oils”. They are available commercially as “Miglyol® 812” (Sasol) or “Capmul® MCT” (Abitec corp.).

Lutein is synthesized by plants and found in high quantities in green leafy vegetables such as spinach and kale. Lutein chemical structure is:

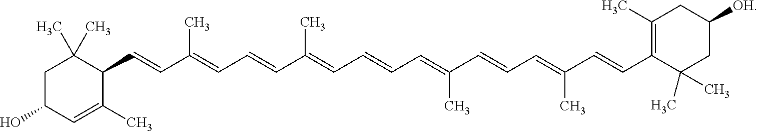 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

Zeaxanthin (CAS registry number 144-68-3) also known as (3R,3′R)-β,β-Carotene-3,3′-diol has the following chemical structure:

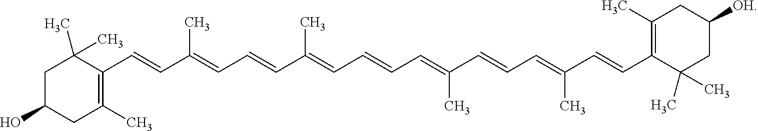 [SMILES: CC1=C(/C=C/C(C)=C/C=C/C(C)=C/C=C/C=C(C)/C=C/C=C(C)/C=C/C2=C(C)C[C@@H](O)CC2(C)C)C(C)(C)C[C@H](O)C1]

Zeacanthin is present in corn, saffron, wolfberries.

The term “6mount” or “therapeutically effective amount” refers to an amount of a drug effective to treat a disease or disorder in a mammal. In the case of age-related macular degeneration (AMD), the effective amount of the drug can reduce or prevent vision loss. For AMD therapy, efficacy in vivo can, for example, be measured by one or more of the following: assessing the mean change in the best corrected visual acuity (BCV A) from baseline to a desired time, assessing the proportion of subjects who lose fewer than 15 letters in visual acuity at a desired time compared with baseline, assessing the proportion of subjects who gain greater than or equal to 15 letters in visual acuity at a desired time compared with baseline, assessing the proportion of subjects with a visual-acuity Snellen equivalent of 20/2000 or worse at desired time, assessing the NEI Visual Functioning Questionnaire, assessing the size of CNV and amount of leakage of CNV at a desired time, as assessed by fluorescein angiography, etc. A “therapeutic dose” is a dose which exhibits a therapeutic effect on the patient and a subtherapeutic dose is a dose which does not exhibit a therapeutic effect on the patient treated.

An “intraocular neovascular disease” is a disease characterized by ocular neovascularization. Examples of intraocular neovascular diseases include, but are not limited to, e.g., proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, etc.

In a particular embodiment of the invention, the present invention would show a synergy of a combination of a N-(4-tert-Butyl-benzyl)-3-chloro-N-[2-(4-chloro-3-ethyl-phenyl)-ethyl]-2-fluoro-5-trifluoromethyl-benz-amide with carotenoid(s), more particular with xanthophyll(s) such as lutein, transzeaxanthin, meso-zeaxanthin and astaxanthin either alone or in combination thereof in the prevention, treatment, delaying progression and/or reduction of eye diseases such as cataract, corneal clouding (opacification), age-related macular degeneration (AMD), in particular AMD.

In a particular embodiment of the invention, the present invention shows a synergy of a combination of N-(4-tert-Butyl-benzyl)-3-chloro-N-[2-(4-chloro-3-ethyl-phenyl)-ethyl]-2-fluoro-5-trifluoromethyl-benz-amide with carotenoid(s), more particular with xanthophyll(s) such as lutein, transzeaxanthin, meso-zeaxanthin and astaxanthin either alone or in combination thereof in the prevention, treatment, delaying progression and/or reduction of age-related macular degeneration (AMD).

Unless otherwise stated all percentages are given in weight percent of the total weight of the composition.

In a particular embodiment, N-(4-tert-Butyl-benzyl)-3-chloro-N-[2-(4-chloro-3-ethyl-phenyl)-ethyl]-2-fluoro-5-trifluoromethyl-benzamide also known as a compound of formula I

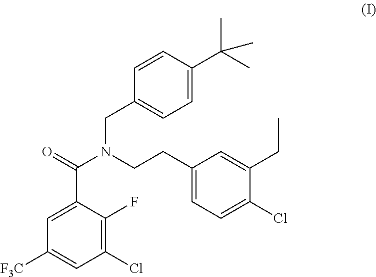 [SMILES: CCc1cc(CCN(Cc2ccc(C(C)(C)C)cc2)C(=O)c2cc(C(F)(F)F)cc(Cl)c2F)ccc1Cl]

N-(4-tert-Butyl-benzyl)-3-chloro-N-[2-(4-chloro-3-ethyl-phenyl)-ethyl]-2-fluoro-5-trifluoromethyl-benzamide, as well as methods of making and using the compound, are described in WO 2007/051714, WO 2008/074677 or WO2011/000793.

N-(4-tert-Butyl-benzyl)-3-chloro-N-[2-(4-chloro-3-ethyl-phenyl)-ethyl]-2-fluoro-5-trifluoromethyl-benz-amide is a potent inhibitor of cholesteryl ester transfer protein (CETP), which was originally developed for the treatment of dyslipidemia and prevention of cardiovascular events. The clinical development was discontinued in 2009 due to strategic portfolio decision; the discontinuation was not due to the safety profile of the product. N-(4-tert-Butyl-benzyl)-3-chloro-N-[2-(4-chloro-3-ethyl-phenyl)-ethyl]-2-fluoro-5-trifluoromethyl-benz-amide has been shown to increase high density lipoprotein-cholesterol (HDL-C) and decrease low density lipoprotein-cholesterol (LDL-C) in humans, and to efflux cholesterol from cells via scavenger receptor SRB1. N-(4-tert-Butyl-benzyl)-3-chloro-N-[2-(4-chloro-3-ethyl-phenyl)-ethyl]-2-fluoro-5-trifluoromethyl-benz-amide is a stable, white to off-white powder, practically insoluble in aqueous media, and soluble in common organic solvents. Liquid-filled hard capsules in the dose strength of 60 mg have been developed.

In vitro, N-(4-tert-Butyl-benzyl)-3-chloro-N-[2-(4-chloro-3-ethyl-phenyl)-ethyl]-2-fluoro-5-trifluoromethyl-benz-amide inhibits CETP activity with an IC50 of 8 nM (4 ng/mL) and no significant binding has been detected at a concentration of 10 μM when tested for 50 pharmacologically relevant receptors. In vivo, inhibition of CETP activity has been demonstrated in hamsters and cynomolgus monkeys.

The pharmacokinetic (PK) properties of N-(4-tert-Butyl-benzyl)-3-chloro-N-[2-(4-chloro-3-ethyl-phenyl)-ethyl]-2-fluoro-5-trifluoromethyl-benz-amide were assessed in the rat and cynomolgus monkey. N-(4-tert-Butyl-benzyl)-3-chloro-N-[2-(4-chloro-3-ethyl-phenyl)-ethyl]-2-fluoro-5-trifluoromethyl-benz-amide showed low total systemic clearance, low volume of distribution, medium to long terminal half-life, relatively low absolute oral bioavailability and a very high plasma protein binding across species. Gastrointestinal absorption was limited, probably by the low solubility and low dissolution rate of this highly lipophilic compound and, resulted in relatively high variability in exposure. In vitro, plasma protein binding was extremely high and concentration-independent. The very high protein binding, in combination with moderate membrane permeability, is the likely cause for the low volume of distribution observed in vivo.

In the rat and cynomolgus monkey, the major excretion route was the bile and feces, independent of the route of administration (approximately 92% of administered dose eliminated). Less than 1% of the dose was eliminated renally.

Two clinical studies have been completed with N-(4-tert-Butyl-benzyl)-3-chloro-N-[2-(4-chloro-3-ethyl-phenyl)-ethyl]-2-fluoro-5-trifluoromethyl-benz-amide in male healthy subject and male mildly dyslipidemic subjects.

Following oral administration of N-(4-tert-Butyl-benzyl)-3-chloro-N-[2-(4-chloro-3-ethyl-phenyl)-ethyl]-2-fluoro-5-trifluoromethyl-benz-amide at single ascending doses (SAD) and multiple ascending doses (MAD) in a first clinical study, a lag time (T_{lag}) of 0.5 hour was observed, and Cₘₐₓ was achieved on average at 3 hours post-dose. AUC₀₋₂₄ₕ and Cₘₐₓ increased in a dose-proportional manner up to single doses of 360 mg (part 1 SAD) and up to multiple doses of 180 mg (part 2 MAD), after which the increase was less than dose-proportional.

A biphasic elimination profile was observed after both single and multiple dosing with a mean terminal half-life ranging between 15 and 35 hours after multiple doses above 10 mg. No accumulation was apparent across doses with a once-daily regimen, except at the highest dose (420 mg), where the accumulation ratio for AUC₀₋₂₄ₕ was 1.4 when comparing Day 14 to Day 1.

Food increased the oral bioavailability of N-(4-tert-Butyl-benzyl)-3-chloro-N-[2-(4-chloro-3-ethyl-phenyl)-ethyl]-2-fluoro-5-trifluoromethyl-benz-amide. A standard breakfast increased exposure in terms of AUC and Cₘₐₓ by approximately 4- to 6-fold, compared with the fasted state and depending on the formulation used. A high-fat breakfast had an additional effect on exposure of approximately 2- to 3-fold above a standard breakfast.

In the combined SAD/MAD study, dose-dependent inhibition of plasma CETP activity was achieved for N-(4-tert-Butyl-benzyl)-3-chloro-N-[2-(4-chloro-3-ethyl-phenyl)-ethyl]-2-fluoro-5-trifluoromethyl-benz-amide. Complete CETP inhibition was observed at the first measurement (2 hours post-dose) with all doses of N-(4-tert-Butyl-benzyl)-3-chloro-N-[2-(4-chloro-3-ethyl-phenyl)-ethyl]-2-fluoro-5-trifluoromethyl-benz-amide in healthy and mildly dyslipidemic subjects on Day 1. The main difference between the different doses was the duration of the inhibition, which was sustained for 24 hours with the two highest tested multiple dose levels (180 mg and 420 mg). The effects on CETP inhibition appeared to reach a plateau at multiple daily doses of 180 mg, as no additional effects were observed with the higher dose of 420 mg. In addition, a dose-dependent and time-dependent increase in CETP mass was observed after multiple doses of N-(4-tert-Butyl-benzyl)-3-chloro-N-[2-(4-chloro-3-ethyl-phenyl)-ethyl]-2-fluoro-5-trifluoromethyl-benz-amide, without evidence of having reached a plateau. After multiple doses of N-(4-tert-Butyl-benzyl)-3-chloro-N-[2-(4-chloro-3-ethyl-phenyl)-ethyl]-2-fluoro-5-trifluoromethyl-benz-amide for 14 days, a similar trend in CETP inhibition was observed.

The CETP inhibition achieved with multiple doses of up to 420 mg N-(4-tert-Butyl-benzyl)-3-chloro-N-[2-(4-chloro-3-ethyl-phenyl)-ethyl]-2-fluoro-5-trifluoromethyl-benz-amide for 14 days in healthy subjects was associated with increase of HDL-C and apolipoprotein A-I (apoA-I) and decrease in LDL-C and apolipoprotein B-100 (apoB-100). For HDL-C, an increase was observed in healthy and mildly dyslipidemic subjects after doses from approximately 30 mg up to 420 mg. The maximum mean increase from baseline ranged from 48% to 55% with the 180 and 420 mg doses, respectively, and was maintained over the entire dosing interval of 24 hours. For LDL-C, plasma levels decreased after 14 days of treatment with all N-(4-tert-Butyl-benzyl)-3-chloro-N-[2-(4-chloro-3-ethyl-phenyl)-ethyl]-2-fluoro-5-trifluoromethyl-benz-amide doses from 10 to 420 mg. A plateau of the effect was achieved with the 180 mg dose and higher doses, with mean LDL-C decreases of at least 30% over the entire dosing interval.

In an embodiment N-(4-tert-Butyl-benzyl)-3-chloro-N-[2-(4-chloro-3-ethyl-phenyl)-ethyl]-2-fluoro-5-trifluoromethyl-benz-amide is a solid in crystalline or amorphous form, more preferably in crystalline form. In a particular embodiment N-(4-tert-Butyl-benzyl)-3-chloro-N-[2-(4-chloro-3-ethyl-phenyl)-ethyl]-2-fluoro-5-trifluoromethyl-benz-amide is in crystalline form A.

In another embodiment, the invention comprises N-(4-tert-Butyl-benzyl)-3-chloro-N-[2-(4-chloro-3-ethyl-phenyl)-ethyl]-2-fluoro-5-trifluoromethyl-benz-amide for use as therapeutic active substances for the treatment, the prophylaxis, delaying progression and/or reduction of eye diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In a particular embodiment, the present invention comprises a pharmaceutical composition comprising the N-(4-tert-Butyl-benzyl)-3-chloro-N-[2-(4-chloro-3-ethyl-phenyl)-ethyl]-2-fluoro-5-trifluoromethyl-benz-amide and a medium chain triglyceridesuseful for the prevention, treatment, delaying progression, and/or reduction of eyes diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In another embodiment of the present invention relates to composition comprising a N-(4-tert-Butyl-benzyl)-3-chloro-N-[2-(4-chloro-3-ethyl-phenyl)-ethyl]-2-fluoro-5-trifluoromethyl-benz-amide, in association with one or more carotenoid(s), in particular wherein the carotenoids are selected from xanthophylls, more particular wherein one carotenoid is lutein, most particularly wherein the carotenoids are lutein and one stereoisomer of zeaxanthin (more particularly zeaxanthin), useful for the prevention, treatment, delaying progression, and/or reduction of eye diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

Said composition is a nutraceutical composition or a pharmaceutical composition useful in particular for the prevention, treatment, delaying progression, and/or reduction of eye diseases such proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In another particular embodiment, the invention relates N-(4-tert-Butyl-benzyl)-3-chloro-N-[2-(4-chloro-3-ethyl-phenyl)-ethyl]-2-fluoro-5-trifluoromethyl-benz-amide which is useful in the treatment of eye diseases such proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In another particular embodiment, the invention relates N-(4-tert-Butyl-benzyl)-3-chloro-N-[2-(4-chloro-3-ethyl-phenyl)-ethyl]-2-fluoro-5-trifluoromethyl-benz-amide which is useful in delaying progression of eye diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In another embodiment, the invention comprises a N-(4-tert-Butyl-benzyl)-3-chloro-N-[2-(4-chloro-3-ethyl-phenyl)-ethyl]-2-fluoro-5-trifluoromethyl-benz-amide for use as therapeutic active substances for the treatment of eye diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In another embodiment, the invention comprises a N-(4-tert-Butyl-benzyl)-3-chloro-N-[2-(4-chloro-3-ethyl-phenyl)-ethyl]-2-fluoro-5-trifluoromethyl-benz-amide for use as therapeutic active substances for delaying progression of eye diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In a particular embodiment, the present invention comprises a pharmaceutical composition comprising N-(4-tert-Butyl-benzyl)-3-chloro-N-[2-(4-chloro-3-ethyl-phenyl)-ethyl]-2-fluoro-5-trifluoromethyl-benz-amide and medium chain triglyceride, useful for the prevention, treatment, delaying progression, and/or reduction of eye diseases, such as cataract, corneal clouding (opacification), age-related macular degeneration (AMD), in particular AMD.

In another embodiment of the present invention, the composition as described therein is a pharmaceutical composition useful in particular for the treatment of eye diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

In another embodiment of the present invention, the composition as described therein is a pharmaceutical composition useful in particular for delaying progression of eye diseases such as proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, retinal neovascularization, in particular diabetic retinopathy, such as diabetic macular edema, retinal occlusion, cataract, corneal clouding (opacification), age-related macular degeneration (AMD), in particular cataract, corneal clouding (opacification), age-related macular degeneration (AMD), more particularly AMD (Wet or Dry), most particularly dry AMD.

The term “therapeutic dose” in this context means that the compound(s) produce(s) a change in the symptoms or conditions associated with the disease or condition which is being treated. It is sufficient that a therapeutic dose produce an incremental change in the symptoms or conditions associated with the disease; a cure or complete remission of symptoms is not required. One having ordinary skill in this art can easily determine whether a dose is therapeutic by establishing criteria for measuring changes in symptoms or conditions of the disease being treated and then monitoring changes in these criteria according to known methods. External physical conditions, histologic examination of affected tissues in patients or the presence or absence of specific cells or compounds, associated with a disease may provide objective criteria for evaluating therapeutic effect. In one example, methods of the invention may be used to treat AMD where therapeutic effect is assessed by changes in preventing vision loss. Other indicators of therapeutic effect will be readily apparent to one having ordinary skill in the art and may be used to establish efficacy of the dose.

The doses may be administered according to any time schedule which is appropriate for treatment of the disease or condition. For example, the dosages may be administered on a daily, weekly, biweekly or monthly basis in order to achieve the desired therapeutic effect and reduction in adverse effects. The dosages can be administered before, during or after the development of the disorder. The specific time schedule can be readily determined by a physician having ordinary skill in administering the therapeutic compound by routine adjustments of the dosing schedule within the method of the present invention. The time of administration of the number of first individual and second individual doses as well as subsequent dosages is adjusted to minimize adverse effects while maintaining a maximum therapeutic effect. The occurrence of adverse effects can be monitored by routine patient interviews and adjusted to minimize the occurrence of side effects by adjusting the time of the dosing. For example, doses may be administered on a daily schedule.

N-(4-tert-Butyl-benzyl)-3-chloro-N-[2-(4-chloro-3-ethyl-phenyl)-ethyl]-2-fluoro-5-trifluoromethyl-benz-amide, which are useful in the prevention, treatment, delaying progression and/or reduction of eye diseases such as cataract, corneal clouding (opacification), age-related macular degeneration (AMD), can also be used in association with other active components. For example, compounds of formula (I) can be used in association with anti VEGF compounds.

In the context of the present specification, by “in association with” it should be understood a co-administration, or a combination of two active principles. The coadministration can be simultaneous, almost simultaneous, or delayed in time by a few days or weeks, for example by up to 4 or 5 weeks.

Vascular endothelial growth factor (VEGF) is an endogenous molecule involved in a number of physiological processes, including blood vessel growth at the foetal stage, during injury healing, or for the growth of new vessels in tissues that have a deficient blood supply. VEGF is also involved in pathological processes, like the development of tumour blood vasculature which allows for growth and spread of the tumour, or the formation of new blood vessels in the eye that eventually contributes to vision loss. Anti VEGF therapies therefore aim to prevent this abnormal blood vessel formation by blocking VEGF action.

Examples of anti-VEGF compounds include Macugen® (Pegaptanib sodium), Lucentis (ranibizumab), Avastatin® (bevacizumab) RhuFab, or VEGF Trap Eye. Therefore, in another aspect, N-(4-tert-Butyl-benzyl)-3-chloro-N-[2-(4-chloro-3-ethyl-phenyl)-ethyl]-2-fluoro-5-trifluoromethyl-benz-amide is used in association with antiVEGF compounds for the prevention, treatment and/or reduction of age-related macular degeneration. In another embodiment, the present invention provides a N-(4-tert-Butyl-benzyl)-3-chloro-N-[2-(4-chloro-3-ethyl-phenyl)-ethyl]-2-fluoro-5-trifluoromethyl-benz-amide and one or more carotenoids, in particular wherein the carotenoid are xanthophylls, more particularly wherein the carotenoid are lutein with optionally one stereosisomer of zeaxanthin for the prevention, the treatment, delaying progression and/or the reduction of eye diseases.

In an embodiment, the at least one zeaxanthin isomer is selected from the group consisting of zeaxanthin and mesozeaxanthine. In a further embodiment, the amount of lutein ranges from 0.5 to 25 mg, and the amount of at least one zeaxanthin isomer ranges from 0.1-5 mg. Advantageously, lutein and at least one zeaxanthin isomer are present in the composition in a ratio lutein:zeaxanthin isomer of about 5:1. In a preferred embodiment, the association is an intimate mixture of a CETP modulator, lutein and at least one zeaxanthin isomer.

The N-(4-tert-Butyl-benzyl)-3-chloro-N-[2-(4-chloro-3-ethyl-phenyl)-ethyl]-2-fluoro-5-trifluoromethyl-benz-amide or the composition according to the present invention is for treating mammal (i.e. cat, dog, hamster, rabbit, mouse, gerbil, rat, Guinea pig, or human, especially a human (i.e. a male or female human).

Accordingly, the invention provides a method for the treatment or prophylaxis of eyes diseases in a mammal, which method comprises administering to a mammal (preferably a mammal in need thereof) a therapeutically effective amount of the pharmaceutical composition. The mammal preferably is a human (i. e., a male or female human). The human can be of any race (e. g., Caucasian or Oriental). The eye diseases is preferably selected from degenerative disease or damage to the retina caused by a disease or an injury, eye strain, accommodative dysfunction of the eye, asthenopia, diabetic retinopathy or dry eye syndrome, the latter caused by either tear or oil gland inflammation. In particular, the method comprises administering a therapeutically effective amount of the composition to an individual to benefit the vision of an individual suffering from eye damage caused by disease or injury or to prevent such disease in man.

In particular embodiments of the present invention, the compositions described herein are pharmaceutical compositions.

The pharmaceutical composition can be, for example, in the form of a pill, capsule or tablet, each containing a predetermined amount of N-(4-tert-Butyl-benzyl)-3-chloro-N-[2-(4-chloro-3-ethyl-phenyl)-ethyl]-2-fluoro-5-trifluoromethyl-benz-amide and in particular coated for ease of swallowing, in the form of a powder or granules. In particular, the pharmaceutical composition is in the form of a capsule comprising N-(4-tert-Butyl-benzyl)-3-chloro-N-[2-(4-chloro-3-ethyl-phenyl)-ethyl]-2-fluoro-5-trifluoromethyl-benz-amide and medium chain triglycerides. For oral administration, fine powders or granules may contain diluting, dispersing and/or surface active agents and may be present, for example, in capsules or sachets in the dry state, or in tablets wherein binders and lubricants may be included. Components such as sweeteners, flavoring agents, preservatives, suspending agents, thickening agents, and/or emulsifying agents also may be present in the pharmaceutical composition.

In a particular embodiment, the composition herein is enclosed in a softgel. More particularly the composition as herein described will comprised 180 mg of N-(4-tert-Butyl-benzyl)-3-chloro-N-[2-(4-chloro-3-ethyl-phenyl)-ethyl]-2-fluoro-5-trifluoromethyl-benz-amide and at least 200 mg of medium chain triglycerides.

In certain embodiments of the present invention, the composition comprises 30 mg to 600 mg of N-(4-tert-Butyl-benzyl)-3-chloro-N-[2-(4-chloro-3-ethyl-phenyl)-ethyl]-2-fluoro-5-trifluoromethyl-benz-amide. In particular, the composition comprises 30 mg to 180 mg of N-(4-tert-Butyl-benzyl)-3-chloro-N-[2-(4-chloro-3-ethyl-phenyl)-ethyl]-2-fluoro-5-trifluoromethyl-benz-amide. More particularly, the composition comprises 60 mg to 180 mg of N-(4-tert-Butyl-benzyl)-3-chloro-N-[2-(4-chloro-3-ethyl-phenyl)-ethyl]-2-fluoro-5-trifluoromethyl-benz-amide. Most particularly, the composition comprises 60 mg to 180 mg of N-(4-tert-Butyl-benzyl)-3-chloro-N-[2-(4-chloro-3-ethyl-phenyl)-ethyl]-2-fluoro-5-trifluoromethyl-benz-amide.

In another embodiment, the composition comprises for pediatric use 15 mg to 90 mg of N-(4-tert-Butyl-benzyl)-3-chloro-N-[2-(4-chloro-3-ethyl-phenyl)-ethyl]-2-fluoro-5-trifluoromethyl-benz-amide. In particular the pediatric composition comprises 30 mg to 90 mg of N-(4-tert-Butyl-benzyl)-3-chloro-N-[2-(4-chloro-3-ethyl-phenyl)-ethyl]-2-fluoro-5-trifluoromethyl-benz-amide.

To be effective, N-(4-tert-Butyl-benzyl)-3-chloro-N-[2-(4-chloro-3-ethyl-phenyl)-ethyl]-2-fluoro-5-trifluoromethyl-benz-amide must be absorbed into the blood. Oral dosing of N-(4-tert-Butyl-benzyl)-3-chloro-N-[2-(4-chloro-3-ethyl-phenyl)-ethyl]-2-fluoro-5-trifluoromethyl-benz-amide is preferred because to be effective N-(4-tert-Butyl-benzyl)-3-chloro-N-[2-(4-chloro-3-ethyl-phenyl)-ethyl]-2-fluoro-5-trifluoromethyl-benz-amide must be taken on a regular basis, such as daily.

The N-(4-tert-Butyl-benzyl)-3-chloro-N-[2-(4-chloro-3-ethyl-phenyl)-ethyl]-2-fluoro-5-trifluoromethyl-benz-amide can be administered to the mammal at any suitable dosage (e.g., to achieve a therapeutically effective amount). For example, a suitable dose of a therapeutically effective amount of compound of formula I for administration to a patient will be between approximately 30 mg to about 600 mg per day. In Particular, a desirable dose is about 60 mg to about 420 mg per day. A preferred dose is about 60, 120 or 180 mg per day. Particularly the daily does would be 180 mg per day.

In another embodiment the invention provides a kit comprising N-(4-tert-Butyl-benzyl)-3-chloro-N-[2-(4-chloro-3-ethyl-phenyl)-ethyl]-2-fluoro-5-trifluoromethyl-benz-amide as described herein, lutein and optionally with one or more stereoisomer of zeaxathin. In a more particular embodiment the invention provides a kit comprising a N-(4-tert-Butyl-benzyl)-3-chloro-N-[2-(4-chloro-3-ethyl-phenyl)-ethyl]-2-fluoro-5-trifluoromethyl-benz-amide as described herein, lutein and optionally with one or more stereoisomer of zeaxathin., prescribing information also known as “leaflet”, a blister package or bottle (HDPE or glass) and a container. The prescribing information preferably includes the advice to a patient regarding the administration of the N-(4-tert-Butyl-benzyl)-3-chloro-N-[2-(4-chloro-3-ethyl-phenyl)-ethyl]-2-fluoro-5-trifluoromethyl-benz-amide (e.g. compound of formula (I) with food, especially to improve the bioavailability of the CETP modulator).

In another embodiment the invention provides a kit comprising a composition as described herein, lutein and optionally with one or more stereoisomer of zeaxathin. In a more particular embodiment the invention provides a kit comprising a composition as described herein, lutein and optionally with one or more stereoisomer of zeaxathin., prescribing information also known as “leaflet”, a blister package or bottle (HDPE or glass) and a container. The prescribing information preferably includes the advice to a patient regarding the administration of N-(4-tert-Butyl-benzyl)-3-chloro-N-[2-(4-chloro-3-ethyl-phenyl)-ethyl]-2-fluoro-5-trifluoromethyl-benz-amide (e.g. compound of formula (I) with food, especially to improve the bioavailability of the CETP modulator).

In another embodiment, the invention provides a kit comprising a composition comprising a therapeutically effective amount of N-(4-tert-Butyl-benzyl)-3-chloro-N-[2-(4-chloro-3-ethyl-phenyl)-ethyl]-2-fluoro-5-trifluoromethyl-benz-amide, lutein and optionally with one or more stereoisomer of zeaxathin., prescribing information, a blister package or bottle and a container. In particular embodiment the invention provides the kit as described herein, wherein the prescribing information includes the advice to a patient regarding the administration of N-(4-tert-Butyl-benzyl)-3-chloro-N-[2-(4-chloro-3-ethyl-phenyl)-ethyl]-2-fluoro-5-trifluoromethyl-benz-amide with food.

In another embodiment, the invention provides a kit comprising a composition comprising a therapeutically effective amount of N-(4-tert-Butyl-benzyl)-3-chloro-N-[2-(4-chloro-3-ethyl-phenyl)-ethyl]-2-fluoro-5-trifluoromethyl-benz-amide, medium chain triglycerides, lutein and optionally with one or more stereoisomer of zeaxathin., prescribing information, a blister package or bottle and a container.

In another embodiment, the invention provides a tablet comprising the composition as herein described.

In another embodiment, the invention provides a composition as herein described for preparing a medicament for the treatment or prevention of eye diseases, such as cataract, corneal clouding (opacification), age-related macular degeneration (AMD), in particular wherein the N-(4-tert-Butyl-benzyl)-3-chloro-N-[2-(4-chloro-3-ethyl-phenyl)-ethyl]-2-fluoro-5-trifluoromethyl-benz-amide is administered at a daily dose of 20 mg to 600 mg, particularly 30 mg to 420 mg, more particularly 30 mg, 60 mg, 80 mg, 90 mg, 180 mg, most particularly 180 mg, particularly wherein N-(4-tert-Butyl-benzyl)-3-chloro-N-[2-(4-chloro-3-ethyl-phenyl)-ethyl]-2-fluoro-5-trifluoromethyl-benz-amide is administered with food.

Other features and embodiments of the invention will become apparent from the following examples which are given for illustration of the invention rather than for limiting its intended scope.

### EXAMPLE 1

The utility of N-(4-tert-Butyl-benzyl)-3-chloro-N-[2-(4-chloro-3-ethyl-phenyl)-ethyl]-2-fluoro-5-trifluoromethyl-benz-amide as medical agents useful in the prevention, treatment, delaying progression and/or reduction of eye diseases, in particular age-related macular degeneration in mammals (e.g. humas) is demonstrated by the activity of the compounds of this invention in conventional essays and the clinical protocol described.

Effect N-(4-tert-Butyl-benzyl)-3-chloro-N-[2-(4-chloro-3-ethyl-phenyl)-ethyl]-2-fluoro-5-trifluoromethyl-benz-amide on the treatment of AMD.

This study 5 is a multicenter, randomized, double-masked, placebo-controlled, parallel study of 18 months' duration in intermediate AMD patients, as defined previously. Approximately 126 patients (randomized 1:1 to the two treatment arms), to reach approximately 100 patients in the PP population will be recruited. Randomization will be stratified for vitamin supplement use (YES/NO), statin use (YES/NO) and age (≦70/>70 years).

Patients who have met all of the eligibility criteria during the screening will be randomized in a 1:1 ratio to 180 mg of N-(4-tert-Butyl-benzyl)-3-chloro-N-[2-(4-chloro-3-ethyl-phenyl)-ethyl]-2-fluoro-5-trifluoromethyl-benz-amide or placebo. During the treatment period, patients will visit the clinical site at randomization, one month and at three months after randomization, and thereafter every three months, with a last dosing visit at 18 months. During the follow-up period patients will visit the clinical site one and four months after last study medication intake. The end of the trial is defined as the last visit of the last patient (LPLV) in the trial.

In addition to sparse PK sampling performed in all patients, a sub-group of around 26 patients will be included in a “PK sub-study” and will include additional plasma PK sampling.

Furthermore, the exploratory efficacy outcome of pigmentary changes (as measured by macular pigment optical density “MPOD”) and scotopic microperimetry sensitivity will be obtained only from subsets of the enrolled patients based on availability of the measurement devices at the study site.

The total duration of the study for each patient will be up to 23 months divided in three phases:
1) Screening and baseline
   Both visits should occur as close as possible and never further than 1 month apart Day −1 (for baseline);
2) Study Drug Administration (Double-blind treatment period)
   Ambulatory visits at clinical sites
   Frequency: Month 1-Day 1; Month 1-Day 28; Month 3-Day 28; Month 6-Day 28; Month 9-Day 28; Month 12-Day 28; Month 15-Day 28; Month 18-Day 28 (single day visit only). Allowing a time-window of ±5 days.
3) Follow-upMonth
   19-Day 28 and Month 22-Day 28. All specified days may vary between ±5 days.

Patients may be admitted to the clinical research unit or stay overnight at an organized facility on Day −1 and will be discharged approximately five hours after study drug administration on Day 1 to ensure that specified assessments could be performed prior to drug administration on Day 1.

The end of the study is defined as the date when the last study patient last observation (LSLO) occurs. LSLO is expected to occur 22 months after the last study patient has been enrolled or four months after last study medication intake.

Target population for this study will be patients with intermediate AMD, as defined in the above presented classification. These patients are at high risk of progressing to late stages of AMD. On the other hand these patients have usually good VA but marked impairments in their dark adaptation function. Therefore, they represent an ideal target population where potential change in dark adaptation function and drusen area/volume changes can be detected. As this population is at high risk to progress to the advanced forms of AMD, reduction of progression is another important endpoint that will be analyzed.

The dose selected for the current study is 180 mg N-(4-tert-Butyl-benzyl)-3-chloro-N-[2-(4-chloro-3-ethyl-phenyl)-ethyl]-2-fluoro-5-trifluoromethyl-benz-amide administered within 15 min of breakfast completion in a once daily (QD) regimen. In other words, half of the enrolled patients will take liquid-filled hard capsules in the dose strength of 60 mg of N-(4-tert-Butyl-benzyl)-3-chloro-N-[2-(4-chloro-3-ethyl-phenyl)-ethyl]-2-fluoro-5-trifluoromethyl-benz-amide, for a single oral administration of 180 mg (three capsules) per day for 18 months wheras the second half of the enrolled patient will take single oral dose administration (three capsules) per day of matching placebo formulation for 18 months.

One of the primary objective of this study is to evaluate the effects of N-(4-tert-Butyl-benzyl)-3-chloro-N-[2-(4-chloro-3-ethyl-phenyl)-ethyl]-2-fluoro-5-trifluoromethyl-benz-amide on change in dark adaptation function after 18 months of treatment.

The dark adaptation test generates a dark adaptation curve. The rod-intercept (DA-RI) is a parameter estimated from the fitting of this curve. DA-RI is the main parameter of interest in this study.

DA-RI is characteristically the rod-mediated dark adaptation function, at the exclusion of cone-mediated dark adaptation. Rod intercept is defined as the time from the bleaching challenge until reaching a sensitivity threshold of 5×10-3 cd/m2.

The primary efficacy parameter of this trial is the difference between treated and placebo arm in mean change from baseline to the month 18 visit in the DA-RI end point.

Estimated treatment effect with 95% CI will be the main parameter of interest for the interpretation of the study results. Statistical test of the null hypothesis of no difference between treatment group will also be reported.

The primary analysis will use mean change from baseline in DA-RI as a continuous variable. MMRM analysis will be used to evaluate the difference between treatment and placebo at the month 18 visit. DA-RI measurement taken at or after the date of incidence of CNV in study or fellow eye will be excluded from the analysis because the effect of CNV treatment in either eye on the DA-RI measure is not well understood.

Additional supportive analysis of the primary endpoint may be conducted. In particular, these could include analyses using a categorized transformation of the change from baseline in DA-RI. For example, the repeatability coefficient and/or a cutoff of 3.1 minute might be used to categorize change from baseline in DA-RI. In the categorical analysis, incidence of CNV in the study eye will be categorized as “progression” or “not-responder”. Incidence of CNV in the fellow eye will be excluded from the analysis.

Two secondary endpoints are being looked at, drusen volume (DV) and Low Luminance Visual Acuity (LLVA). The analysis of the key secondary endpoints will follow the same logic as outlined above, with the main parameter of interest being the difference between treated and placebo arm in mean change from baseline to the month 18 visit.

DV will be analyzed using similar approach as the primary endpoint of DA-RI. For statistical analysis and reporting purposes, the cube root of DV will be used. For the analysis of change from baseline in DV as a continuous variable, measurements after incidence of CNV or GA in the study eye will be excluded, because progression to CNV or GA are sometimes associated with a decrease in DV, which could introduce a bias in the analysis. Change from baseline in DV may also be analyzed as a categorical variable. For example, the coefficient of repeatability and/or 50% of the baseline cube root DV may be used as cut-off on a patient per patient basis. Patients with progression to CNV or GA in study eye will be included in this analysis and will be categorized as “progression” (or non-responder) irrespective of actual DV measurement.

LLVA will be analyzed using similar approach as the primary endpoint of DA-RI. Low Luminance Deficit (LLD) defined as LLD=(BCVA-LLVA) will be used as a primary parameter to assess low luminance visual acuity function.

### EXAMPLE 2

### Determination of Viability in HUVECs in Response of CETP Inhibitors

Material

Human umbilical vein endothelial cells (HUVEC), the growth medium EGM and the basal medium EBM was obtained from Lonza. Additional FCS for the starving medium was used form Invitrogen.

The viability assay with alamarBlue® from Invitrogen was done in 96 well gelatin coated plates from BD.

The tested compounds were dissolved in DMSO at a stock concentration of 10 mM.

The tested compound were: Torcetrapib, Anacetrapib, Evacetrapib and N-(4-tert-Butyl-benzyl)-3-chloro-N-[2-(4-chloro-3-ethyl-phenyl)-ethyl]-2-fluoro-5-trifluoromethyl-benz-amide (compound 1)

Torcetrapib, [2R, 4S] 4-[(3, 5-bis-trifluoromethyl-benzyl)-methoxycarbonyl-amino]-2-ethyl-6-trifluoromethyl-3, 4-dihydro-2H-quinoline-1-carboxylic acid ethyl ester, can be prepared according to the procedure as disclosed in WO2000017164 or WO2001040190.

Anacetrapib, (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one, can be prepared according to the procedure as disclosed in WO2006/014357, WO2006/014413 or WO2007005572.

Evacetrapib, Trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl) cydohexanecarboxylic acid can be prepared according to the procedure as disclosed in WO06002342 or WO2011002696.

VEGF-A₁₆₅ was obtained from R&D Systems® and has been dissolved at a stock concentration of 100 ug/ml in PBS.

Method:

HUVECs cells were maintained in Endothial growth medium (EGM) until Passage 5. For the viability assay HUVECs have been seeded in EBM with 0.5% FCS on gelatin coated 96 well plates at a cell density of 11.000 cells/well. Outer wells were left free and have been filled with medium afterwards. Cells were incubated overnight in a cell culture incubator at 37° C. with 5% CO2. Compounds and VEGF have been diluted in EBM/0.5% FCS to a 20× working concentration. 5 ul of the 20× pre-dilution have been added to the cells. HUVECs which have to be intended to grow under hypoxia conditions have been transferred to a cell culture incubator with 1% O2, 5% CO2 and 37° C. HUVECs grown under normoxia conditions were incubated in a normal cell culture incubator. After 72 h, 11 ul of alamarBlue® have been added to each well of the cells subsequently incubated for 4 h in the cell culture incubator. The absorbance has been detected at 570 nm with a reference wavelength of 600 nm.

Results:

A dose dependent survival signal was determined in response to VEGF-A₁₆₅ in HUVECs. This outcome is more visible under normoxia than under hypoxia conditions.

TABLE 2
Results from alamarBlue ® assay after treatment with VEGF for 72 h
under normoxia and hypoxia conditions (viability measured at 570 nm)

| | |
|---|---|
| VEGF | Condition |

| [ng/ml] | normoxia | hypoxia |
|---|---|---|
| 1.2 | 0.0268 | 0.0480 |
| 2.3 | 0.0394 | 0.0679 |
| 4.7 | 0.0582 | 0.0983 |
| 9.4 | 0.0908 | 0.1114 |
| 18.8 | 0.1265 | 0.1039 |
| 37.5 | 0.1449 | 0.0927 |
| 75 | 0.1642 | 0.0906 |
| 150 | 0.1618 | 0.0825 |

Under normoxia conditions a positive effect of viability in HUVECs cells is seen when cells were treated with the compound 1 and Anacetrapib. For the compounds Torcetrapib and Evacetrapib no influence of viability could be detected.

TABLE 3
Results from alamarBlue ® assay after treatment with CETPi for 72 h
under normoxia (OD 570 nm-600 nm)

| c [nM] | Torcetrapib | Anacetrapib | Evacetrapib | Compound 1 |
|---|---|---|---|---|
| 4.1 | 0.0020 | 0.0096 | 0.0032 | 0.0073 |
| 10.2 | −0.0004 | 0.0170 | 0.0158 | 0.0075 |
| 25.6 | 0.0160 | 0.0203 | 0.0164 | 0.0056 |
| 64 | 0.0007 | 0.0119 | 0.0051 | 0.0062 |
| 160 | 0.0194 | 0.0168 | 0.0227 | 0.0127 |
| 400 | 0.0152 | 0.0352 | 0.0103 | 0.0223 |
| 1000 | 0.0001 | 0.0662 | 0.0191 | 0.0600 |
| 2500 | 0.0138 | 0.0846 | −0.0107 | 0.1512 |

When HUVECs are treated with CETP inhibitors (CETPi) under hypoxic conditions only compound 1 induced a strong cell survival. Anacetrapib also show a positive effect on viability, albeit with lower efficacy, whereas Torcetrapib and Evacetrapib had negative effect on the cell viability of HUVECs.

TABLE 4
Results from alamarBlue ® assay after treatment with CETPi for 72 h
under hypoxia (OD 570 nm-600 nm)

| c [nM] | Torcetrapib | Anacetrapib | Evacetrapib | Compound 1 |
|---|---|---|---|---|
| 4.1 | 0.0129 | 0.0003 | 0.0141 | −0.0059 |
| 10.2 | 0.0200 | 0.0065 | 0.0129 | 0.0152 |
| 25.6 | 0.0118 | 0.0014 | 0.0131 | 0.0144 |
| 64 | 0.0111 | 0.0093 | 0.0076 | 0.0118 |
| 160 | 0.0137 | −0.0022 | 0.0168 | 0.0233 |
| 400 | 0.0308 | 0.0260 | 0.0403 | 0.0326 |
| 1000 | −0.0388 | 0.0565 | 0.0564 | 0.0967 |
| 2500 | −0.0784 | 0.0814 | −0.1084 | 0.1488 |

### EXAMPLE 3

XRPD patterns of N-(4-tert-Butyl-benzyl)-3-chloro-N-[2-(4-chloro-3-ethyl-phenyl)-ethyl]-2-fluoro-5-trifluoromethyl-benzamide crystalline form A were recorded at ambient conditions in transmission geometry with a STOE STADI P diffractometer (Cu K alpha radiation source, primary monochromator, position sensitive detector, angular range 3° to 42° 2Theta, approximately 30 minutes total measurement time). The samples were prepared and analyzed without further processing (e.g. grinding or sieving) of the substance.

| | relative |
|---|---|
| 2theta/° | intensity/% |
| 5.4 | 22.5 |
| 7.0 | 85.4 |
| 9.1 | 17.6 |
| 10.2 | 18.6 |
| 12.1 | 19.4 |
| 14.6 | 100.0 |
| 17.5 | 32.8 |
| 19.2 | 25.9 |
| 19.5 | 25.1 |
| 19.9 | 38.0 |
| 20.6 | 21.9 |
| 21.8 | 25.2 |
| 22.1 | 22.2 |
| 22.6 | 37.3 |
| 23.2 | 40.9 |
| 25.8 | 19.3 |

1-27. (canceled)

28. A method of treating, preventing, retarding or ameliorating an eye disease, the method comprising administering a N-(4-tert-Butyl-benzyl)-3-chloro-N-[2-(4-chloro-3-ethyl-phenyl)-ethyl]-2-fluoro-5-trifluoromethyl-benzamide to a subject in need thereof.

29. The method of claim 28, wherein the eye disease is proliferative retinopathies, choroidal neovascularization, age-related macular degeneration, diabetic and other ischemia-related retinopathies, diabetic macular edema, pathological myopia, von Hippel-Lindau disease, histoplasmosis of the eye, Central Retinal Vein Occlusion, corneal neovascularization, or retinal neovascularization.

30. The method of claim 28, wherein the eye disease is cataract, corneal clouding, age-related macular degeneration, or dry AMD.

31. The method of claim 28, wherein the method further comprises administering one or more carotenoids to the subject.

32. The method of claim 31, wherein the carotenoids comprise xanthopylls.

33. The method of claim 21, wherein the carotenoids comprise zeaxanthin.

34. The method of claim 31, wherein the carotenoids comprise lutein.

35. The method of claim 28, wherein the method further comprises administering an anti-VEGF compound to the subject.

36. The method of claim 35, wherein the anti-VEGF compound is Macugen, Lucentis, Avastatin.

37. The method of claim 28, wherein the N-(4-tert-Butyl-benzyl)-3-chloro-N-[2-(4-chloro-3-ethyl-phenyl)-ethyl]-2-fluoro-5-trifluoromethyl-benzamide is of form A.

38. A pharmaceutical composition comprising N-(4-tert-Butyl-benzyl)-3-chloro-N-[2-(4-chloro-3-ethyl-phenyl)-ethyl]-2-fluoro-5-trifluoromethyl-benz-amide and medium chain triglyceride.

39. A pharmaceutical composition comprising N-(4-tert-Butyl-benzyl)-3-chloro-N-[2-(4-chloro-3-ethyl-phenyl)-ethyl]-2-fluoro-5-trifluoromethyl-benz-amide and an anti-VEGF compound.

40. The pharmaceutical composition of claim 39, wherein the anti-VEGF compound is Macugen, Lucentis, Avastatin.

41. The pharmaceutical composition of claim 39, wherein the N-(4-tert-Butyl-benzyl)-3-chloro-N-[2-(4-chloro-3-ethyl-phenyl)-ethyl]-2-fluoro-5-trifluoromethyl-benzamide is of form A.

42. A pharmaceutical composition comprising N-(4-tert-Butyl-benzyl)-3-chloro-N-[2-(4-chloro-3-ethyl-phenyl)-ethyl]-2-fluoro-5-trifluoromethyl-benz-amide and one or more cartenoids.

43. The method of claim 42, wherein the carotenoids comprise xanthopylls.

44. The method of claim 42, wherein the carotenoids comprise zeaxanthin.

45. The method of claim 42, wherein the carotenoids comprise lutein.

46. The pharmaceutical composition of claim 42, wherein the N-(4-tert-Butyl-benzyl)-3-chloro-N-[2-(4-chloro-3-ethyl-phenyl)-ethyl]-2-fluoro-5-trifluoromethyl-benzamide is of form A.
